(19) United States
(12) Patent Application Publication
(10) Pub. No.: US 2004/0160221 A1
(43) Pub. Date: Aug. 19, 2004

(54) Method to excite planar slices in a magnetic resonance tomography device, accounting for nonlinear gradient fields

(76) Inventors: Berthold Kiefer, Erlangen (DE); Robert Krieg, Nuemberg (DE)

Correspondence Address: SCHIFF HARDIN, LLP, PATENT DEPARTMENT, 6600 SEARS TOWER, CHICAGO, IL, 60606-6473, US

(21) Appl. No.: 10/699,290

(22) Filed: Oct. 31, 2003

(30) Foreign Application Priority Data
Oct. 31, 2002 (DE) 10250913.1

(51) Int. Cl. G01V 3/00; A61B 5/055

(52) U.S. Cl. 324/307; 324/309; 600/410

(57) ABSTRACT

In a method and MRT device for planning and generating planar MRT slices based on deformation-free corrected MRT overview images, the MRT device having a nonlinear gradient system with data with regard to the nonlinear variations being stored, at least one MRT overview image is generated, MRT overview image is corrected on the basis of the stored data with regard to the nonlinear variations, the ensuing variations of a planar slice selected by the operator in the corrected MRT overview image are determined on the basis of the stored data with regard to the nonlinear variations, an MR sequence with a radio-frequency excitation pulse as well as gradient pulses is calculated, such that, given simultaneous radiation of these pulses, the slice to be acquired undergoes a curvature in the exposure that is inverse to the nonlinear variations of the MRT overview image, and data for the planar slice are acquired using the calculated MR sequence.

### BACKGROUND OF THE INVENTION

1. Field of the Invention

The present invention concerns in general magnetic resonance tomography (MRT) as applied in medicine to examine patients. The present invention thereby concerns in particular a method to excite, by the use of spatially-selective gradient pulses, straight slice packets in the spatial domain based on non-planar distorted overview images generated due to nonlinear gradient fields.

2. Description of the Prior Art

MRT is based on the physical phenomenon of nuclear magnetic resonance and has been successfully used as an imaging method for over 15 years in medicine and biophysics. In this examination modality, the subject is exposed to a strong, constant magnetic field. The nuclear spins of the atoms in the subject, which were previously randomly oriented, thereby align. Radio-frequency energy can now excite these “ordered” nuclear spins to a specific oscillation. This oscillation generates in MRT the actual measurement signal that is acquired by means of appropriate reception coils. By the use of inhomogeneous magnetic fields generated by gradient coils, the signals from the measurement subject can be spatially coded in all three spatial directions, known as “spatial coding”.

The acquisition of the data ensues in MRT in what is known as k-space (frequency domain). The MRT image in the image domain is linked with the MRT data in k-space by means of Fourier transformation. The spatial coding of the subject, which is present throughout k-space, ensues in all three spatial directions by means of gradients. Differentiation is made between the slice readout (establishes an acquisition slice in the subject, typically the z-axis), the frequency coding (establishes a direction in the slice, typically the x-axis) and the phase coding (determines the second dimension within the slice, typically the y-axis).

Thus, a slice is first selectively excited, for example in the z-direction. The coding of the spatial information in the slice ensues by combined phase and frequency coding by both of these (already mentioned) orthogonal gradient fields, that in the example of a slice excited in the z-direction are generated by the (likewise already mentioned) gradient fields in the x-direction and the y-direction.

A first possible sequence for acquiring the data in an MRT measurement is shown in FIGS. 2a and 2b. The sequence used is a spin-echo sequence. In this, the magnetization of the spin is rotated in the x-y plane by a 90° excitation pulse. In the course of time (½ T_{E}; T_{E} is the echo time) there occurs a dephasing Δφ of the magnetization portions that mutually form the transverse magnetization in the x-y plane M_{xy}. After a certain time (for example, ½ T_{E}), a 180° pulse is emitted in the x-y plane such that the dephased magnetization components are flipped without that precession direction and precession speed of the individual magnetization portions being changed. After a further time duration ½ T_{E}, the magnetization components again point in the same direction, i.e. it amounts to a regeneration of the transverse magnetization designated as a “rephasing”, The complete regeneration of the transverse magnetization is designated as a spin-echo.

In order to measure an entire slice of the subject to be examined, the imaging sequence is repeated n-times for different values of the phase coding gradient, for example G^{y}, with the frequency of the nuclear magnetic resonance signal (spin-echo signal) in each sequence passing through the Δt-clocked analog-digital converter ADC being scanned, digitized, and stored N-times in equidistant time steps Δt, in the presence of the readout gradient G^{x}. In this manner, numerical matrix (matrix in k-space or k-matrix) is generated line by line with NxN data points, as shown in FIG. 2B. A symmetrical matrix with NxN points Is only one example; asymmetrical matrices or other k-space arrangements can be generated. The path followed fro scanning the data entries in the k-space matrix is called the k-space trajectory. Four further possibilities for a two-dimensional k-space scanning are graphically represented in FIGS. 3A-3D. FIG. 3A represents a radially symmetric spoke-shaped, k-space trajectory, FIG. 3B represents a spiral-shaped k-space trajectory, FIG. 3C represents a pin wheel-shaped k-space trajectory, and FIG. 3D represents a Lissajous figure-shaped k-space trajectory. From such data sets in k-space, MR images of the slice in question with a resolution of NxN pixels can be directly reconstructed by Fourier transformation. The scanning density of the respective trajectory, in the cases of FIG. 3A and FIG. 3D, is not uniform, and, as explained below, must be taken into account by a suitable factor in the Fourier transformation,

The different versions of k-space scanning can be differentiated as follows:

a) in the data readout: the manner by which the gradients, in particular the readout gradient, are switched during the readout of the MR signal, and

b) in the slice excitation: the manner by which the gradients, in particular the slice selection gradient, are switched during the radiation of the radio-frequency excitation pulse.

The slice excitation, like the readout of the MR signal, must be completed in an amount of time that corresponds to the decay of the transverse-magnetization. Otherwise, the different lines of the k-matrix would be differently weighted corresponding to the order of their being filled with data entries: specific spatial frequencies would be overemphasized, others would be underemphasized. High measurement speeds, however, put extremely high demands on the gradient system. In practice, gradient amplitudes of approximately 25 mT/m are used. To reverse the polarity (switch) of the gradient field, significant energy must be converted in the shortest possible time; for example, the switch times are in the range of ≦0.3 ms. The time in which the maximum gradient amplitude can be achieved is generally designated as a gradient rise time (slew rate). The slew rate is in practice the speed with which a gradient field can be activated. Older systems have or had slew rates of 20-40 mT/ms. Modem devices exhibit slew rates of 100-200 mT/ms, which in modem devices means the respective gradient fields exhibit strong nonlinearity, due to the gradient coil inductance (explained in more detail below).

Nonlinearities of the gradient fields generally produce a deformation of the reconstructed MR images that is not desired in most cases. In modern MRT devices, such deformations can be corrected in the image display. The correction predominantly serves for cosmetic purposes and does not increase the precision of the diagnosis. MR device operators nevertheless consider this deformation correction as desirable, in particular when the MRT images are forwarded to other specialist doctors, who are not knowledgeable as to the details of magnetic resonance tomography, because such persons otherwise may think that the images are flawed.

If the device operator wishes to plan further measurements by positioning further slice packets on such a corrected image, a conflict arises. Because the planned slice packets, with regard to their spatial position in reality, “see” nonlinear gradient fields, the image plane that is planned based on the corrected image does not correspond to the actual acquired image plane.

Nonlinearity and slew rate are directly connected with one another. For example, there are MRT devices with gradient systems that can (for technical application reasons) be operated selectively in two states (modes). The gradient system is designed such that, in a first operating state (mode 1), a large but not strong gradient field can be generated with a relatively slow or moderate gradient rise time. Such a gradient field is, as a rule, very linear. In a second operating state (mode 2), a relatively small but strong gradient field can be generated with a faster gradient rise time. Such a generated gradient field is, as a rule, strongly nonlinear.

If, in mode 1, a first slice packet is not acquired as an overview image, on the basis of which further slice exposures are to be planned, but is acquired in mode 2, a conflict again arises as above; the, planned slice planes do not coincide with the already acquired image plane, due to the different nonlinearity of the gradient fields.

### SUMMARY OF THE INVENTION

An object of the present invention, for the planning of further MRT measurements on corrected MRT images, is to provide a method and an MRT device to implement the method, within which conflicts as described above are prevented in a simple manner.

This object is achieved according to the invention in a method for planning and generating planar MRT slices based on deformation-free corrected MRT overview images using an MRT device that has a nonlinear gradient system with data with regard to the nonlinear variation being stored, including the following steps:

generation of at least one MRT overview image,

correction of the at least one MRT overview image on the basis of the stored data with regard to the nonlinear variations,

determination of the ensuing variations of a planar slice, selected by the operator in the corrected MRT overview image, based on the stored data with regard to the nonlinear variations,

calculation of an MR sequence with a radio-frequency excitation pulse as well as gradient pulses, such that, given simultaneous emission of these pulses, the slice to be acquired undergoes a curvature in the exposure that is inverse to the nonlinear variations of the MRT overview image,

acquisition of the data file for the planar slice by application of the calculated MR sequence.

The data with regard to the nonlinear variations, as a rule are exactly measured one time before the delivery of the device to the installation site.

The data with regard to the nonlinear variations, can be stored in the system computer and/or in the sequence control.

The calculation of the radio-frequency excitation pulse, as well as the gradient pulses, ensues according to a known method to calculate spatially selective pulses.

The above object also is achieved in accordance with the invention by a magnetic resonance tomography device that implements the inventive method.

### DESCRIPTION OF THE DRAWINGS

FIG. 1 is a schematic block diagram of a magnetic resonance tomography device operable in accordance with the invention.

FIG. 2A schematically illustrates the time curve of the gradient pulse sequence for a spin-echo sequence.

FIG. 2B schematically illustrates the temporal sampling of the k-matrix in a spin-echo sequence.

FIGS. 3A, 3B, 3V and 3D respectively schematically illustrate four variants of k-space scanning.

FIG. 4 schematically shows possible gradient pulse forms as well as the simultaneously emitted radio-frequency pulses for excitation of defined slices in the spatial domain.

FIG. 5A schematically shows an MRT image with deformations in the upper image region and lower image region, and a planned acquisition slice.

FIG. 5B schematically shows the same MRT exposure from FIG. 5A after a deformation correction with a curve of the planned exposure slice corresponding to the deformation.

FIG. 5C schematically shows the same MRT exposure from FIG. 5B with a target slice precisely corresponding to the reflected curved exposure slice.

### DESCRIPTION OF THE PREFERRED EMBODIMENTS

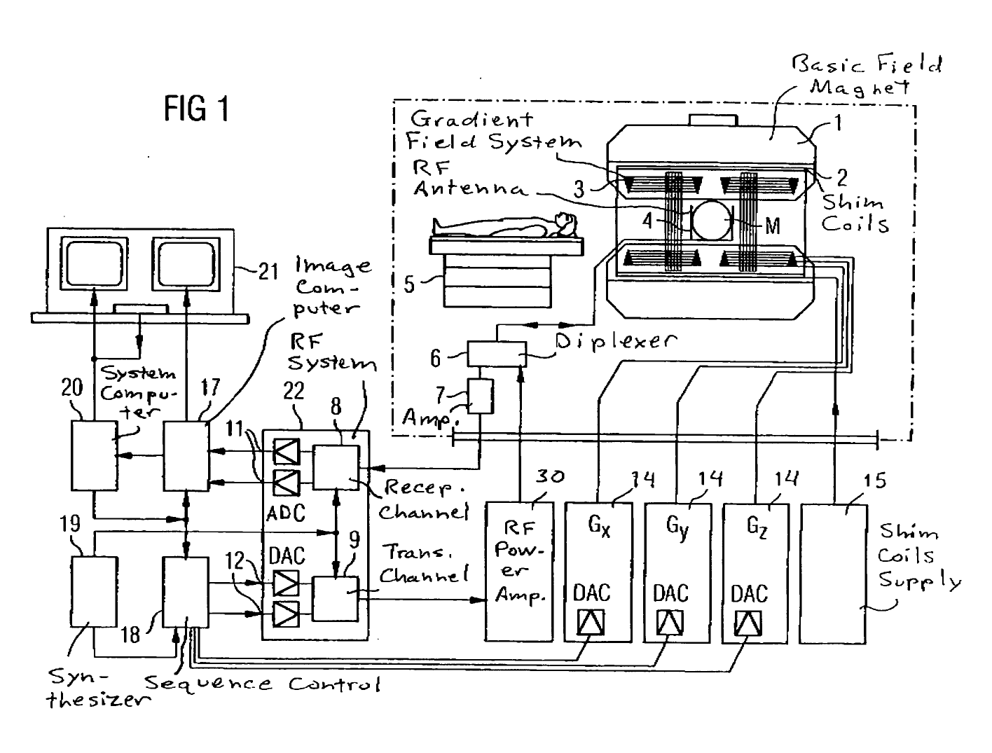

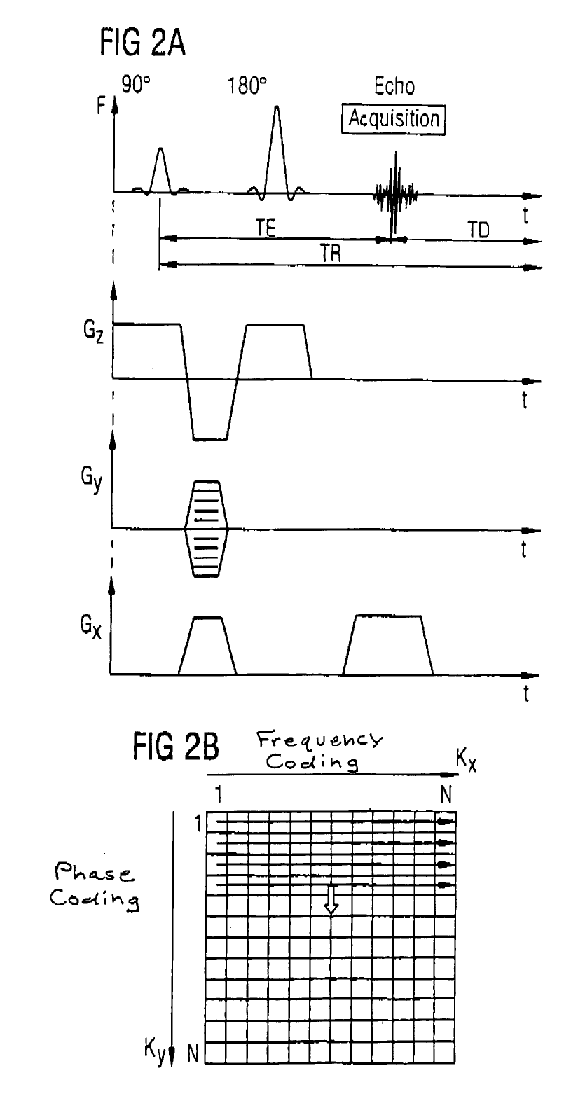

FIG. 1 is a schematic illustration of a magnetic resonance tomography device that generates gradient pulses according to the present invention. The assembly of the components of the magnetic resonance tomography device corresponds to the assembly of a typical tomography device, but the device differs from conventional devices in the operation described below. A basic field magnet 1 generates a temporally constant strong magnetic field to polarize or align the nuclear spins in the examination region of a subject, such as for example a part of a human body to be examined. The high homogeneity of the basic field magnet required for the nuclear magnetic resonance measurement is defined in a spherical measurement volume M, into which the parts of the human body to be examined are moved. To support the homogeneity requirements, and in particular to eliminate temporally invariable influences, shim plates made of ferromagnetic material are mounted at appropriate locations. Temporally variable influences are eliminated by shim coils 2 that are controlled by a shim power supply 15.

A cylindrical gradient coil system 3 is used in the basic field magnet 1 that has three coils. The coils are supplied with power by respective amplifiers 14 to generate respective linear gradient fields in the directions of the Cartesian coordinate system. The first coil of the gradient field system 3 generates a gradient Gₓ in the x-direction, the second coil of the gradient field system 3 generates a gradient G_{y} in the y-direction, and the third coil of the gradient field system 3 generates a gradient G_{z} in the z-direction. Each amplifier 14 has a digital-analog converter that is controlled by a sequence control 18 for timed generation of the gradient pulses.

A radio-frequency antenna 4 is located within the gradient field system 3 that converts the radio-frequency pulse emitted by a radio-frequency power amplifier 30 into a magnetic alternating field to excite the nuclei and align the nuclear spins of the subject to be examined or of the region of the subject to be examined. The alternating field originating from the precessing nuclear spins, meaning as a rule the nuclear spin echo signals ensuing from a pulse sequence composed of one or more radio-frequency pulses and one or more gradient pulses, is converted by the radio-frequency antenna 4 into a voltage that is supplied via an amplifier 7 to a radio-frequency reception channel 8 of a radio-frequency system 22. The radio-frequency system 22 furthermore has a transmission channel 9 in which the radio-frequency pulses are generated for the excitation of the nuclear magnetic resonance. The respective radio-frequency pulses are digitally represented in the sequence control 18 as a sequence of complex numbers based on a pulse sequence predetermined by the system computer 20. This number sequence is supplied via respective inputs 12 as a real part and an imaginary part to a digital-analog converter in the radiation detector system 22, and supplied from this to a transmission channel 9. In the transmission channel 9, the pulse sequences are modulated by a radio-frequency carrier signal whose base frequency corresponds to the resonance frequency of the nuclear spins in the measurement volume.

The changeover from transmission operation to reception operation ensues via a transmission-reception diplexer 6. The radio-frequency antenna 4 emits the radio-frequency pulses to excite the nuclear spins in the measurement volume M and samples resulting echo signals. The correspondingly acquired magnetic resonance signals are phase-sensitively demodulated in the reception channel 8 of the radio-frequency system 22 and converted via respective analog-digital converter into a real part and an imaginary part of the measurement signal. An image is reconstructed by an image computer 17 from the measurement data acquired in such a manner. The administration of the measurement data, the image data and the control program ensues with a system computer 20. Based on a specification with control programs, the sequence control 18 monitors the generation of the respective desired pulse sequences and the corresponding scanning of k-space. In particular, the sequence control 18 controls the timed switching of the gradients, the emission of the radio-frequency pulses with defined phase and amplitude, and the reception of the magnetic resonance signals. The time base (clock) for the radio-frequency system 22 and the sequence control 18 is provided by a synthesizer 19. The selection of corresponding control programs to generate a magnetic resonance image, as well as the representation of the generated nuclear magnetic resonance image, ensues via a terminal 21 (console) that includes a keyboard as well as one or more screens.

As mentioned above, the measured MRT images that are displayed on the screen of the terminal 21 are distorted due to the nonlinearity of the gradient fields. This deformation can be corrected by suitable image processing software implemented on the system computer 20 or the sequence controller 18. The basis for such correction programs is a precise knowledge of the nonlinearities obtained by exact measurement of the gradient fields, which is typically implemented once before the delivery of the MRT device. The measured nonlinear variations are then stored as a data set in a storage medium accessible by the system computer 20.

As also discussed above the positioning of further slices based on such corrected MRT images leads to significant conflicts and hinders or limits the operator to a considerable degree in the further measurement planning. For this reason, in MRT devices of various manufacturers slice planning or positioning based on corrected images is generally not allowed, which leads in part to customer complaints. There are also manufacturers that allow a measurement planning based on corrected images by the operator on their MRT devices, but then according to statements by customers, it can occur that a region to be examined is actually missed in the planned slice.

The present invention enables the operator, in spite of a nonlinear gradient system (meaning in spite of an MRT device which generates nonlinear fields), to undertake a reasonable positioning of further slices based on corrected MRT images.

A reasonable planning of exposure slices based on the corrected images presumes that the excitation slices exhibit a definite curvature, which is used to produce the deformation-correcting output Signal so the curvature is compensated in the corrected image. Conversely, in an MRT device whose gradient system generates nonlinear fields, this would require the excitation of straight gradient packets, which is not possible in devices at this time.

From the literature—especially J. Hardy, E. Cline, P. Bottomley, Journal of Magnetic Resonance, 87, 639-645 (1990)—it is theoretically known how, with suitable gradient pulse modulation using what are known as “spatially selective radio-frequency pulses”, in principle any two-dimensional or three-dimensional formation can be excited in the spatial domain.

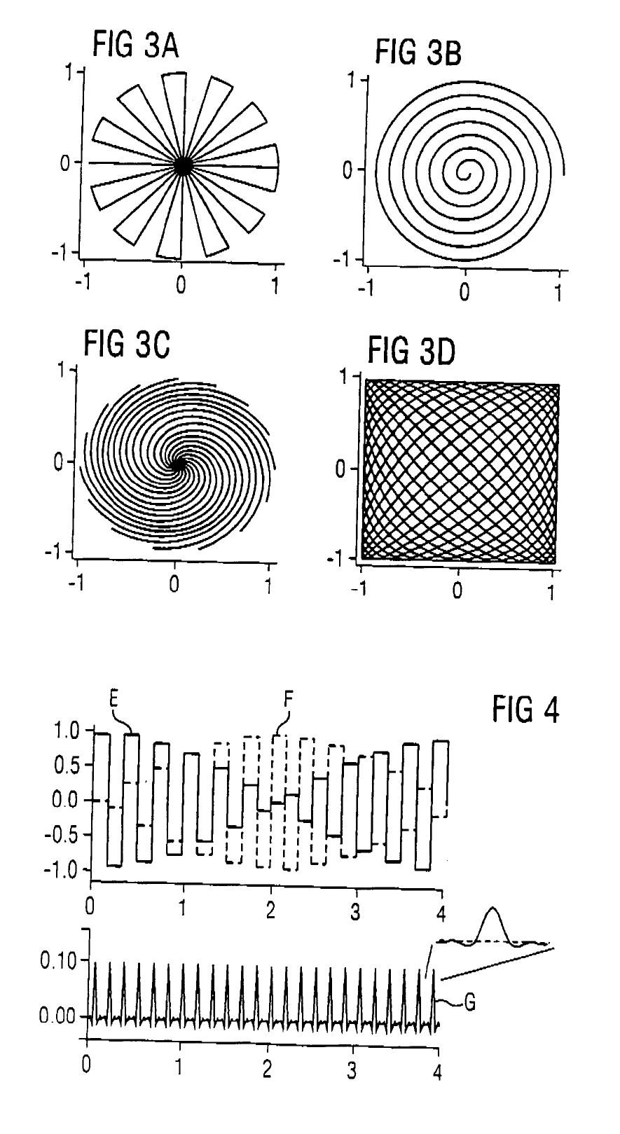

FIG. 4 shows, for example, the necessary curve of the gradients in the x-direction E (solid line) as well as in the y-direction F (dashed line) in order to scan a 24-segment radially symmetric k-space trajectory according to FIG. 3A. The corresponding curve of the simultaneous radio-frequency excitation pulse to be emitted is shown in FIG. 4G. The k-space trajectory determines how the gradients are to be switched during the emission of the radio-frequency excitation pulse. The present invention utilizes such a specific gradient pulse modulation (spatially selective pulses) in order to generate specific slices in the spatial domain.

Thus, in order to excite a straight slice packet in nonlinear gradient fields, spatially selective pulses are inventively used in order to generate a pre-deformation with opposite sign. This “artificially” generated curve of the slice is then exactly cancelled by the nonlinearity of the gradient fields, such that the actual excited slice is straight.

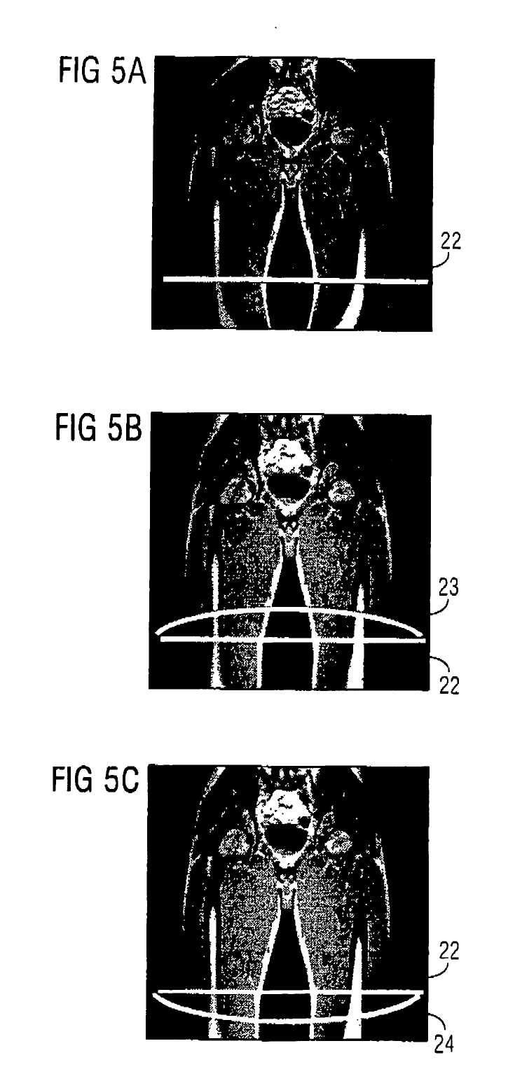

The procedure is explained in more detail using FIGS. 5A through 5C. An MRT exposure from the upper to the lower thigh region is shown in FIG. 5A. As can be clearly seen, the exposure has severe deformations (distortions) in the upper image region and in the lower image region. The plotted straight line 22 represents a planned slice that is also actually addressed in an excitation by slice selection. However—as can be seen—due to the deformation, the anatomy was not imaged planar, meaning in a plane. FIG. 5B shows the exposure from FIG. 5A after a distortion correction. By the equalization of the image, the previously straight slice 22 from now on undergoes a clear curvature (slice 23). If one wanted to excite a planar slice in the now-corrected image—corresponding to slice 22 in FIGS. 5A or 5B-the slice to be excited must exhibit a deformation that compensates the deformation of slice 23 in FIG. 5B. Such a slice is plotted in FIG. 5C as slice 24. This desired slice 24 is exactly the reflection of the slice 23 from FIG. 5B at the planned slice 22. It should be noted that the shown slice curves are only schematic drawings, and the effect of the deformation is shown overemphasized.

For example, the invention can proceed according to an operating protocol as follows:

(1) planning a straight slice on a corrected MRT image,

(2) determining the real ensuing variation of this slice,

(3) calculation of gradients and excitation pulses via whose combined radiation slice variations are generated with opposite signs,

(4) run the sequence.

The determination of the gradient field nonilnearitles for the purpose of distortion correction of further slices to be planned can ensue according to the method specified in German published patent application 19540837. The data determined according to this method specify exactly the deviation of a slice profile from a straight slice and are inventively used as a basis for the calculation of the pulse modulation in step (3) above.

In step (3), excitation and gradient pulses must now be designed such that these, in combination in the cases of linear gradient fields, would excite the slice 24 from FIG. 5C. According to J. Hardy, E. Cline, P. Bottomley, Journal of Magnetic Resonance, 87, 639-645 (1990), such a pulse combination can be calculated as follows: (the formulas likewise originate from this publication:

(a) determine the Fourier transformations of the formation to be excited—here the curved layer 24 in FIG. 5c.

(b) determine a shortest possible k-space trajectory (the curve of which is given by \dot{K}(t)) which covers the surface of the planar determined Fourier transformations. This k-space trajectory determines how the gradients G(t) are to be switched during the radio-frequency pulse:

\overline{G}(t)=(I/γ)\overline{K}(t)

G(t) and K(t) are vector quantities.

(c) simultaneous radiation of a specific gradient pulse with the gradient pulses according to the relationship

$${B}_{i} ( t ) = \frac{- i ( \overline{G} ( t ) )}{ρ ( \overline{K} ( t ) )} {\int }_{- ∞}^{∞} {\int }_{- ∞}^{∞} slice ( {r}^{⇁} ) {ⅇ}^{ⅈ ( \overline{K} ( {r}^{⇁} ) \overline{r}} ⅆ x ⅆ y$$

The Fourier transformations determined in (a) of the slice profile of the slice 24 to be excited are in the integral (FIG. 5C).

The denominator in the factor before the integral is a correction factor that takes into account the density of the k-space trajectory.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for planning and generating a magnetic resonance tomography (MRT) slice of an examination subject, based on a deformation-free, corrected MRT overview image using an MRT device having a nonlinear gradient system and access to stored data representing nonlinear variations associated with the nonlinear gradient system, said method comprising the steps of:
generating an MRT overview image of a subject;
correcting said MRT overview image based on the stored data regarding the nonlinear variations, thereby obtaining a corrected MRT overview image;
in said corrected MRT overview image, selecting a planar slice and determining variations in said planar slice based on the stored data; calculating an MRT pulse sequence, including at least one radio-frequency excitation pulse and at least two gradient pulses, such that upon emission of said at least one radio-frequency excitation pulse and said at least two gradient pulses said planar slice exhibits a curvature that is inverse to the nonlinear variations in the corrected MRT overview image; and acquiring data for said planar slice from the subject by executing said MRT sequence.

2. A method as claimed in claim 1 comprising measuring said data represent nonlinear variations associated with the nonlinear gradient system only once, and storing said data in a memory.

3. A method as claimed in claim 2 comprising storing said data as a dataset in a memory in a processor selected from the group consisting of a system computer of said MRT device and a sequence control of said MRT device.

4. A method as claimed in claim 1 comprising calculating said at least one radio-frequency excitation pulse and said at least two gradient pulses in said MRT sequence according to a method for calculating spatially selected pulses.

5. A magnetic resonance tomography (MRT) apparatus comprising:
an MRT scanner for acquiring magnetic resonance data from a subject, said MRT scanner having an RF system. a nonlinear gradient system, and a memory containing stored data representing wherein nonlinear variations associated with the nonlinear gradient system;
a system computer and a sequence control for operating said MRT scanner to generate an MRT overview image of the subject;
a processor having access to said memory for correcting said MRT overview image based on the stored data, thereby obtaining a corrected MRT overview image;
a user interface allowing a user to select, in said corrected MRT overview image, a planar slice, and said processor then determining variations in said planar slice based on the stored data;
said processor calculating an MRT pulse sequence, including at least one radio-frequency excitation pulse and at least two gradient pulses, such that upon emission of said at least one radio-frequency excitation pulse by said RF system and emission said at least two gradient pulses by said nonlinear gradient system cause said planar slice to exhibit a curvature that is inverse to the nonlinear variations in the corrected MRT overview image; and
said MRT scanner acquiring data for said planar slice from the subject by executing said MRT sequence.

6. A magnetic resonance tomography apparatus as claimed in claim 5 comprising wherein said processor is said system computer.

7. A magnetic resonance tomography apparatus as claimed in claim 5 comprising wherein said processor is said sequence control.

8. A magnetic resonance tomography apparatus as claimed in claim 5 wherein said processor calculates said at least one radio-frequency excitation pulse and said at least two gradient pulses in said MRT sequence according to a method for calculating spatially selected pulses.
